(19) United States
(12) Patent Application Publication
(10) Pub. No.: US 2023/0228756 A1
(43) Pub. Date: Jul. 20, 2023

(54) TRANSFER LEARNING ACROSS HEMATOLOGICAL MALIGNANCIES

(71) Applicant: AHEAD Intelligence Ltd., Taipei City (TW)

(72) Inventors: Jeng-Lin Li, Taipei City (TW); Yu-Lin Chen, Taipei City (TW); Chi-Chun Lee, Taipei City (TW); Yu-Fen Wang, Taipei City (TW)

(21) Appl. No.: 18/164,374

(22) Filed: Feb. 3, 2023

Related U.S. Application Data
(63) Continuation of application No. PCT/US21/44390, filed on Aug. 3, 2021.
(60) Provisional application No. 63/060,148, filed on Aug. 3, 2020.

(51) Int. Cl. G01N 33/574; G16H 50/20; G16H 50/50

(52) U.S. Cl. CPC G01N 33/57426; G16H 50/20; G16H 50/50

(57) ABSTRACT

Introduced here is an approach to improving the automatic identification of hematological malignancies by taking advantage of established databases through transfer learning. At a high level, this approach attempts to address the cross-domain gap by preserving knowledge of the source domain for better optimization of the target domain.

### CROSS-REFERENCE TO RELATED APPLICATIONS

This application is a continuation of International Application No. PCT/US2021/044390, filed Aug. 3, 2021, which claims priority to U.S. Provisional Application No. 63/060,148, titled “Transfer Learning Across Hematological Malignancies” and filed on Aug. 3, 2020, which are incorporated by reference herein in their entireties.

### TECHNICAL FIELD

Various embodiments concern computer programs and associated computer-implemented techniques for transferring knowledge across different domains.

### BACKGROUND

Leukemia (occasionally spelled “leukaemia”) are cancers that start in cells that would normally develop into different types of blood cells. Often, leukemias begin in the bone marrow and result in high numbers of abnormal blood cells. These abnormal blood cells may be referred as “leukemia cells” or “blast cells.” The exact cause of leukemia is unknown, so a diagnosis is normally made based on the results of a blood test or bone marrow test (also referred to as a “bone marrow biopsy”). Generally, the blood test or bone marrow biopsy is taken when an individual (also referred to as a “patient” or “subjects”) reports that she is suffering from symptoms such as bleeding, bruising, fatigue, and fever.

There are four main types of leukemia—acute lymphoblastic leukemia (ALL), acute myeloid leukemia (AML), chronic lymphocytic leukemia (CLL), and chronic myeloid leukemia (CML)—as well as a number of less common types. Leukemias belong to a broader group of conditions that affect the blood, bone marrow, and lymphoid system. This broader group of conditions are commonly referred to as “tumors of the hematopoietic and lymphoid tissues.”

The aforementioned types have historically been divided based mainly on (i) whether the leukemia is acute (i.e., fast growing) or chronic (i.e., slow growing) and (ii) whether the leukemia starts in myeloid cells or lymphoid cells. ALL and AML generally start in the bone marrow but then often move into the blood and other parts of the human body, including the lymph nodes, liver, and spleen. The rate at which blast cells (or simply “blasts”) spread through the human body corresponds to whether the underlying leukemia is acute or chronic.

### BRIEF DESCRIPTION OF THE DRAWINGS

FIG. 1 includes a high-level illustration of a framework for employing transfer learning to extend insights into one hematological malignancy to another hematological malignancy.

FIG. 2 includes a high-level illustration of a process by which representations are extracted from underlying data stored in a database.

FIG. 3 includes a high-level illustration of a process by which an analysis platform distills knowledge from a source domain to improve its ability to perform classification in a target domain.

FIG. 4 includes a diagram illustrating how, with the parameters of a pretrained model fixed at stable values, the representations and labels can be leveraged for the target database in training another model.

FIG. 5 includes a schematic flowchart of a process for performing harmonized learning.

FIG. 6 includes a schematic diagram of the framework described above with reference to FIGS. 1-5 in its testing phase for the target domain.

FIG. 7 includes the experimental results for three different classifiers, namely, Logistic Regression, Support Vector Machine, and deep neural network.

FIG. 8 illustrates a network environment that includes an analysis platform.

FIG. 9 includes a flow diagram of a process for improving the classification of hematological malignancies through transfer learning.

FIG. 10 includes a flow diagram of a process for effecting transfer learning by applying more than one trained model to data related to a specimen.

FIG. 11 is a block diagram illustrating an example of a processing system in which at least some operations described herein can be implemented.

Various features of the technology described herein will become clearer to those skilled in the art by studying the Detailed Description in conjunction with the drawings. Certain embodiments are shown in the drawings for the purpose of illustration. However, those skilled in the art will recognize that alternative embodiments may be employed without departing from the principles of the technology. Accordingly, although specific forms of the technology are shown in the drawings, the technology is amenable to various modifications.

### DETAILED DESCRIPTION

To understand leukemia and lymphoma, it helps to understand the blood and lymph systems of the human body.

Bone marrow is the soft inner part of some bones. At a high level, bone marrow is comprised of blood-forming cells, fat cells, and supporting tissues. A small fraction of the blood-forming cells in the bone marrow are normally blood stem cells. Inside the bone marrow, blood stem cells undergo changes in order to develop into red blood cells, platelets, or white blood cells. Red blood cells (RBCs) carry oxygen from the lungs to other tissues into the human body, as well as take carbon dioxide back to the lungs for removal (e.g., via exhalation). Platelets are cell fragments that are made from a type of blood stem cell called a “megakaryocyte.” Platelets are important in plugging holes in blood vessels that are caused by cuts, bruises, and the like. White blood cells (WBCs) are responsible for helping the human body fight off infections.

There are three main types of WBCs—lymphocytes, granulocytes, and monocytes. Lymphocytes are the main cells that make up the lymph tissue found in lymph nodes and other parts of the human body. Lymphocytes develop from calls called “lymphoblasts” to become mature, infection-fighting cells. There are two main types of lymphocytes—B lymphocytes (also referred to as “B cells”) and T lymphocytes (also referred to as “T cells”). B cells help protect the human body by making proteins called antibodies that attach to germs, while T cells generally help destroy those germs. ALL develops from early forms of lymphocytes. ALL can start in early B cells or T cells at early stages of maturity. Lymphoma also starts in the lymphocytes, though it normally affects B cells or T cells in the lymph nodes rather than the blood and bone marrow. Granulocytes are WBCs that contain granules. These granules normally contain enzymes and other substances that may be helpful in destroying germs. There are three types of granulocytes—neutrophils, basophils, and eosinophils—that can be distinguished by the size and color of the granules. Monocytes also help protect the body against bacteria. Normally, monocytes circulate in the bloodstream for a relatively short interval of time (e.g., roughly one day) and then enter the tissues to become macrophages, which can destroy germs by surrounding and then digesting them.

The term “myeloid cell” is normally used to refer to those blood stem cells that can develop into RBCs, platelets, or WBCs other than lymphocytes. In contrast to ALL, these myeloid cells are the ones that are abnormal in the case of AML.

The lymphatic system (also referred to as the “lymphoid system”) is an organ system that is part of the circulatory system and immune system. The lymphoid system is made up of a large network of lymph, lymphatic vessels, lymph nodes, lymphatic organs, and lymphatic tissues. The vessels carry a clear fluid referred to as “lymph” towards the heart. Unlike the cardiovascular system, the lymphatic system is not a closed system. This means that problems affecting the lymphoid system can quickly spread throughout the body without timely treatment.

As mentioned above, leukemia diagnoses are normally made by healthcare professionals based on the results of blood tests or bone marrow tests. By looking at a sample of the blood of an individual, a healthcare professional can determine whether there are abnormal levels of RBCs, platelets, or WBCs—which may suggest leukemia. A blood test could also show the presence of blasts, though not all types of leukemia cause blasts to circulate in the blood. Sometimes blasts stay in the bone marrow. For that reason, the healthcare professional may recommend a bone marrow test in which a sample of the bone marrow is removed in order to look for blasts.

While recent advances in medicine have improved the survival rates of individuals diagnosed with leukemia, unexpected outcomes still abruptly affect the prognosis in some cases. Current clinical practice uses the identification of minimal residual disease (MRD) as a prognosis indicator that is detected using flow cytometry (FC). At a high level, FC is a technique that is used to detect and measure characteristics of a population of cells. In an FC experiment, a sample containing cells is focused—ideally one cell at a time—through a laser beam, where the scattered light is characteristic to the cells. Cells are often labeled with fluorescent markers so light is absorbed and then emitted within a band of wavelengths. Thus, the FC experiment may involve measuring fluorescent excitement on antibody markers to produce high-dimensional data.

Healthcare professionals have historically examined these data, for example, through manual gating on visualized two-dimensional plots, to determine appropriate diagnoses. This approach is not only laborious and time-consuming, but also prone to error since these healthcare professionals must make subjective decisions. While several entities have proposed adapting machine learning (ML) algorithms or artificial intelligence (AI) algorithms for management of FC data, handling massive amounts of data remains a challenge in clinical practices. Acceptable performance by ML and AI algorithms relies on substantial amounts of labeled data. Sufficient amounts of labeled data have not historically been available, however. This hinders the generalizability and applicability of ML and AI algorithms in real-world situations.

Many clinical databases—even those created relatively recently—tend to either possess only a limited number of names or include results related to a single leukemia subtype. This is unlikely to change. In real-world clinical settings, FC data (and the corresponding diagnostic reports) usually requires heavy cleaning or processing. As such, labeled data is unlikely to become easily accessible in meaningful amounts. Moreover, there are several subtypes of hematological malignancies as discussed above, and each subtype may be associated with a different occurrence probability. Collecting a sufficient number of samples and the corresponding diagnostic outcomes for each subtype in a single medical site is likely to be difficult, if not impossible, in most scenarios.

Introduced here, therefore, is an approach to improving the automatic identification of hematological malignancies by taking advantage of established databases through transfer learning. At a high level, this approach attempts to address the cross-domain gap by preserving knowledge of the source domain for better optimization of the target domain. The predictive capabilities of a computer-implemented model (or simply “model”) may be improved through the use of transfer learning.

As further discussed below, this approach to implementing transfer learning may have two steps, namely, a first step in which a source domain model is pre-trained and then a second step in which the source domain model is tuned so as to produce a target domain model. This paradigm (i.e., pre-training and then tuning) can sometimes cause negative knowledge transfer due to the domain gap between these tasks. Historically, there was no way to quantitatively measure this domain gap, which meant that successful transfer learning in this paradigm was often uncontrollable and/or unrealistic. To address this issue, a framework for harmonized learning can be leveraged. Harmonized learning enables the model to correct outputs of another model that is trained using an unclean or unprocessed database. Assume, for example, that the model is based on a neural network with parameters that are either predetermined based on examples or tuned through experimentation/learning. In such a situation, the neural network may be able to automatically correct predictions made by another neural network that is trained with sub-optimal data.

With this approach, the model developed for the target domain may be able to deploy the power of pretraining-tuning paradigm and fill the domain gap through harmonized learning. This can facilitate the generalization of the model towards more heterogenous disease prediction tasks that relate to each other, though with unknown or unmeasurable domain gap. Accordingly, the approach described herein could be used in any transfer learning scenario across different hematological malignancies where sufficient samples (also referred to as “examples”) are available in one disease with a more limited number of samples available for another disease. Thus, the source and target domains could be hematological malignances such as ALL, AML, CLL, CML, Hodgkin lymphoma and non-Hodgkin lymphoma (diffuse large B-cell lymphoma, follicular lymphoma, mantle cell lymphoma, T-cell lymphoma), multiple myeloma, acute erythroid leukemia, and other solid tumors. Accordingly, the source domain may be described as being associated with a first hematological malignancy, and the target domain may be described as being associated with a second hematological malignancy that is different than the first hematological malignancy. While embodiments may be described with reference to particular hematological malignancies (e.g., AML as the source domain and ALL as the target domain), these hematological malignancies were selected for the purpose of illustration.

Embodiments may also be described in the context of executable instructions for the purpose of illustration. However, those skilled in the art will recognize that aspects of the present application could be implemented via hardware, firmware, or software. As an example, a disease analysis platform (or simply “analysis platform”) could be embodied as a computer program that offers support for reviewing information related to the progression and/or status of a hematological malignancy, cataloging treatments, reviewing diagnoses proposed by models, and the like.

### Terminology

References in the present disclosure to “an embodiment” or “some embodiments” mean that the feature, function, structure, or characteristic being described is included in at least one embodiment. Occurrences of such phrases do not necessarily refer to the same embodiment, nor are they necessarily referring to alternative embodiments that are mutually exclusive of one another.

Unless the context clearly requires otherwise, the terms “comprise,” “comprising,” and “comprised of” are to be construed in an inclusive sense rather than an exclusive or exhaustive sense (i.e., in the sense of “including but not limited to”). The term “based on” is also to be construed in an inclusive sense rather than an exclusive or exhaustive sense. Thus, unless otherwise noted, the term “based on” is intended to mean “based at least in part on.”

The terms “connected,” “coupled,” and variants thereof are intended to include any connection or coupling between two or more elements, either direct or indirect. The connection or coupling can be physical, logical, or a combination thereof. For example, elements may be electrically or communicatively coupled to one another despite not sharing a physical connection.

The term “module” may refer broadly to software, firmware, hardware, or combinations thereof. Modules are typically functional components that generate one or more outputs based on one or more inputs. A computer program may include or utilize one or more modules. For example, a computer program may utilize multiple modules that are responsible for completing different tasks, or a computer program may utilize a single module that is responsible for completing all tasks.

When used in reference to a list of multiple items, the term “or” is intended to cover all of the following interpretations: any of the items in the list, all of the items in the list, and any combination of items in the list.

### Overview of Model-Based Framework for Transfer Learning

As mentioned above, the present disclosure generally concerns a model-based framework for transfer learning. This framework may be helpful in facilitating classification (e.g., target domain MRD classification) of a hematological malignancy when a limited amount of data regarding that hematological malignancy is available for training purposes. Said another way, this framework can be used to help intelligently develop a model for classifying a hematological malignancy by incorporating insights learned by another model that is trained to classify another hematological malignancy. As further discussed below, this framework may make use of model parameters (or simply “parameters”) that are learned from a source domain database rather than the source domain data directly. This framework not only improves the predictive performance in the target domain, but also may prevent or inhibit the privacy problems involved in transferring knowledge across different databases.

At a high level, the framework involves leveraging two important concepts that are represented in different steps. First, a knowledge distillation step (also referred to as a “knowledge filtration step”) that aims to condense knowledge from a first database (e.g., an AML database) together with a corresponding model (e.g., an AML MRD classification model). Second, a harmonized learning step that aims to supplement the information loss in the knowledge distillation step. Together, these steps allow for more effective performance than naive pretraining and tuning approaches, especially when combined with ML or AI.

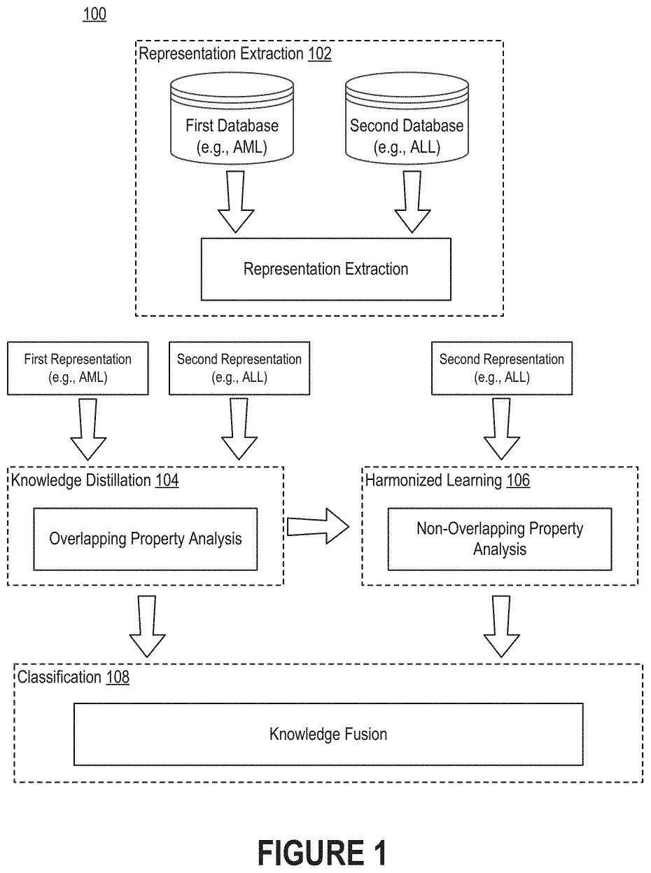

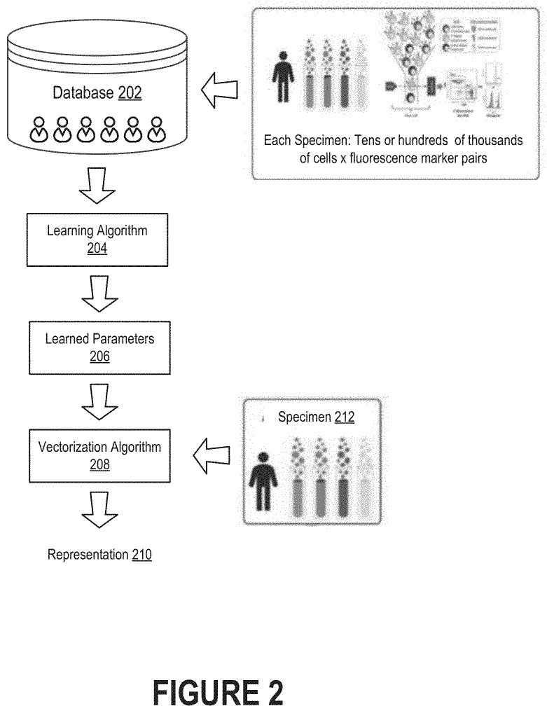

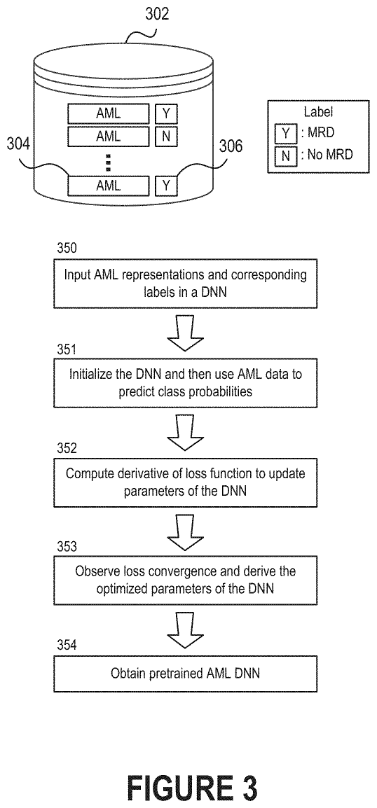

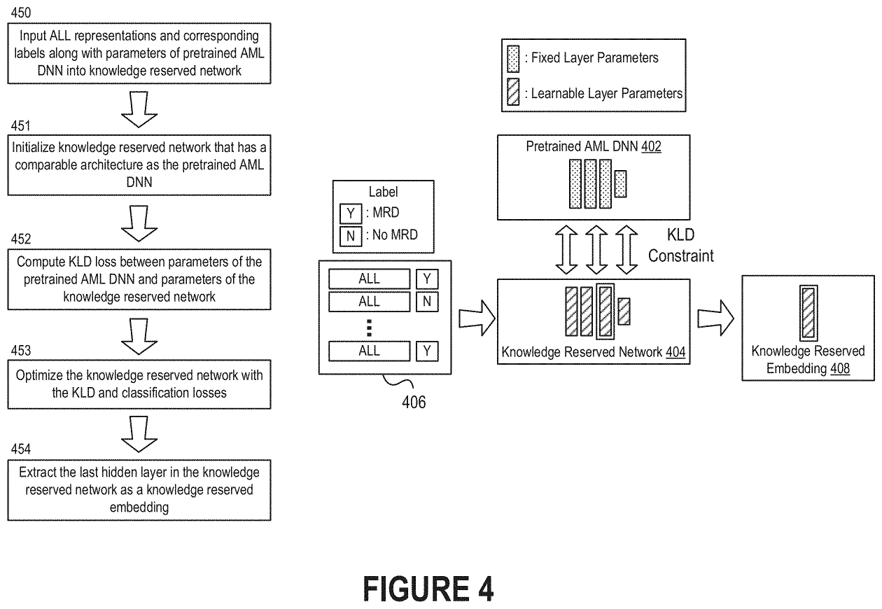

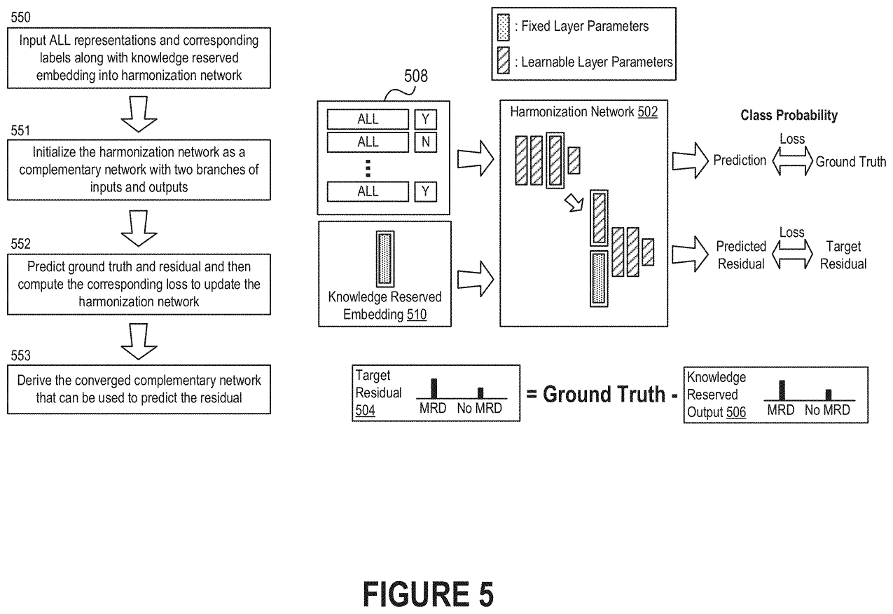

FIG. 1 includes a high-level illustration of a framework 100 for employing transfer learning to extend insights into one hematological malignancy to another hematological malignancy. As shown in FIG. 1, the framework 100 can include various stages. These stages may include a representation extraction stage 102, a knowledge distillation stage 104, a harmonized learning stage 106, and a classification stage 108. The representation extraction stage 102 is further discussed below with reference to FIG. 2, the knowledge distillation stage 104 is further discussed below with reference to FIGS. 3-4, and the harmonized learning stage 106 is further discussed below with reference to FIG. 5.

In the representation extraction stage 102, an analysis platform may derive representations of data obtained from a first database and a second database. Generally, the first and second databases include information regarding different hematological malignancies. As an example, information regarding AML may be stored in the first database, while information regarding ALL may be stored in the second database. In such a scenario, AML may be representative of the source domain for which sufficient information is available, and ALL may be representative of the target domain to which insights learned from the source domain are to be transferred. Representations of the data in the first and second databases may be extracted with, for example, Gaussian mixture models (GMM), Fisher Vectorization, or another ML algorithm.

In some embodiments, the first database and/or the second database is publicly accessible (e.g., via the Internet). For example, the analysis platform may acquire information from the first and second databases by initiating a connection via respective data interfaces (e.g., application programming interfaces). In other embodiments, the first database and/or the second database is privately maintained and managed. For example, the first database may include proprietary clinical data generated by a first healthcare system over time, and the analysis platform may be granted access to the first database in accordance with an agreement between the first healthcare system and an entity that manages the analysis platform. Similarly, the second database may include proprietary clinical data generated by a second healthcare system over time, and the analysis platform may be granted access to the second database in accordance with another agreement between the second healthcare system and the entity that manages the analysis platform. The first healthcare system may be different than the second healthcare system, or the first healthcare system may be the same as the second healthcare system.

The nature of the representation extraction stage 102 may depend on the form of the data in the first and second databases. As an example, if the first and second databases include FC data, each entry may be representative of a data structure that is formatted in accordance with the Flow Cytometry Standard (FCS). FCS is a file format standard for the reading and writing of data from FC experiments. The file format described a file that is a combination of textual data that is followed by binary data, and the order of the file format is normally as follows: (1) header segment, (2) text segment, (3) data segment, (4) optional analysis segment, (5) cyclic redundancy check (CRC) value, and (6) optional other segments.

Together, the knowledge distillation stage 104 and harmonized learning stage 106 may be used to perform classification and obtain class probabilities, as further discussed below. In the knowledge distillation stage 104, the analysis platform may learn the overlapping proprieties between the representations of the data stored in the first and second databases. As an example, if the first database includes information regarding AML and the second database includes information regarding ALL, the analysis platform may seek to learn the properties that overlap between AML and ALL for ALL MRD classification. Thus, the analysis platform may attempt to learn which properties of a first hematological malignancy impact classification for a second hematological malignancy. These overlapping properties may be generally referred to as a “knowledge reserved model.” In the harmonized learning stage 106, the analysis platform may learn the non-overlapping properties between the representations of the data stored in the first and second databases. Referring again to the example above, the analysis platform may learn the ALL properties that do not overlap with the AML properties. These ALL-specifies properties can be thought of as complementary to the knowledge reserved model.

In the knowledge distillation stage 104 and harmonized learning stage 106, which may be driven—partially or entirely—by ML or AI, classification can be performed so as to produce predictions made by the knowledge reserve model and ALL-specific properties, respectively. These predictions may be representative of class probabilities (e.g., for different diagnoses for the target domain—here, ALL). In the classification stage 108, the analysis platform can obtain a final classification output (O) by summing the knowledge reserved out (O_{K}) and residual (R), which are the outputs of the knowledge distillation stage 104 and harmonized learning stage 106, respectively, as shown below:

O=O_{K}+R. Eq. 1

As an example, assume that the analysis platform is tasked with transferring representations learned from analysis of AML data to ALL data for which a lesser number of samples are available. In such a scenario, the analysis platform may obtain FCS files from a first database (also referred to as an “AML database”) that includes entries with information regarding diagnoses of AML and a second database (also referred to as an “ALL database”) that includes entries with information regarding diagnoses of ALL. These FCS files can then be examined by the analysis platform so that representations can be extracted as discussed above with reference to step 102, so as to obtain specimen-level representations for AML and ALL. The analysis platform can then perform knowledge distillation to elicit knowledge from the AML database and have the knowledge preserved in a model trained for ALL MRD classification. Since this joint optimization could miss information existing only in the ALL database, the analysis platform can perform harmonized learning to elicit the residual information that was not captured through knowledge distillation. The final prediction that is produced by the analysis platform may be an output that is representative of the sum of the knowledge reserved network produced through knowledge distillation and the residual produced through harmonized learning.

### A. Representation Extraction

FIG. 2 includes a high-level illustration of a process by which representations are extracted from underlying data stored in a database 202. This process may be performed by an analysis platform as part of a representation extraction step (e.g., representation extraction step 102 of FIG. 1). At a high level, this is the process by which the analysis platform can derive specimen-level representations for entries corresponding to diagnoses of a hematological malignancy. Each entry may correspond to a single diagnosis (and thus a single patient).

As mentioned above, the analysis platform may use a learning algorithm 204 to extract representations of the various specimens included in data stored in a database 202. In FIG. 2, the database 202 includes FCS files that correspond to different specimens tested through experimentation. However, the data stored in the database 202 could be in another format.

The goal of the learning algorithm 204 may be to extract structured information from the database 202 in a consistent manner to ensure that the resulting representations can be compared to those generated for a different database. Normally, the learning algorithm 204 is an unsupervised ML algorithm that can be trained to extract representations. For example, the analysis platform may extract representations with a GMM that is trained using some or all of the FCS files in the database 202, and each specimen may be encoded as a vectorized representation 210 using Fisher scoring based on the learned parameters 206. Said another way, when a specimen 212 is provided as input, the analysis platform can extract a representation 210 by performing vectorization based on the learned parameters 206.

Since each data structure (e.g., FCS file) may correspond to a specimen that contains upwards of tens or hundreds of thousands of cells, this cell-level data may be encoded to the specimen level. As mentioned above, cells are normally labeled with fluorescent markers so light is absorbed and then emitted within a band of wavelengths in an FC experiment. All of the combinations of fluorescent marker pairs may be concatenated at the cell level to train the learning algorithm 204 (e.g., GMM). Through the application of a vectorization algorithm 208 (e.g., that implements Fisher scoring), the analysis platform can produce a fixed dimensional representation vector for each specimen. In the event that the vectorization algorithm implements Fisher scoring, the vectorization algorithm 208 may compute the gradient between each specimen and the learned parameters 206. Since these learned parameters 206 can be thought of as representative of the entire population of the database 202, this approach can be conceptualized to discover, compute, or otherwise establish how much these learned 206 parameters should change to fit a specific specimen. Each representation 210 may embed the relation between the corresponding specimen and the entire population of the database, so as to equip the analysis platform with discriminative capacity.

### B. Knowledge Distillation

FIG. 3 includes a high-level illustration of a process by which an analysis platform distills knowledge from a source domain to improve its ability to perform classification in a target domain. This process may be performed by the analysis platform as part of a knowledge distillation step (e.g., knowledge distillation step 104 of FIG. 1). At a high level, this is the process by which the analysis platform can transfer insights learned in a source dimension corresponding to a first hematological malignancy to a target dimension corresponding to a second hematological malignancy.

The analysis platform can accomplish this by leveraging a learning scheme to incorporate and/or filter knowledge from a classification model for a first hematological malignancy to another classification model for a second hematological malignancy. As an example, the analysis platform may attempt to transfer the knowledge from an AML MRD classification model (also referred to as the “source MRD classification model” or simply “source model”) to another model that is trained to classify MRD based on an analysis of data included in an ALL database. In such a scenario, this other model may be referred to as the “target MRD classification model” or simply “target model.”

In some embodiments, the analysis platform trains a deep neural network (DNN) “from scratch” using a large-scale database associated with a first hematological malignancy (e.g., AML) to classify the presence of MRD as shown in FIG. 3. Those skilled in the art will recognize that the DNN is simply one example of a model that could be used by the analysis platform. The representations 304 from the AML database 302 and corresponding labels 306 indicating whether the corresponding specimens were deemed to have MRD can be fed into the DNN for training purposes. After training is performed with classification loss where parameters are converged and optimized, the analysis platform can indicate this learned network as a pretrained AML DNN. That is, the analysis platform can indicate that this learned network is representative of a pretrained source model.

This pretrained AML DNN may ultimately converge to a set of optimized parameters that can robustly predict MRD in the AML database 302. This AML database 302 is relatively large—which is one reason why training is possible—so this set of optimized parameters may be well tuned for discriminating MRD in AML specimens. As such, if the analysis platform is interested in transferring learning from the AML domain into another domain, the analysis platform may utilize an AML database or another relatively large database with MRD-related information as the source database. Generally, the source database (here, the AML database 302) includes at least 1,000 specimens (˜500 with MRD, ˜500 without MRD). This number of specimens represents an estimated number for a relatively robust population of the source domain, though the source database could include a greater or lesser number of specimens. Similarly, the source database could include a different ratio of “with MRD” versus “without MRD.” Larger diversity is generally better to form a good source domain, though such diversity may not be possible in some instances. The target database, meanwhile, may be any size so long as its data is not well represented. As an example, the target database may have several hundred specimens.

With this pretrained AML DNN, the analysis platform may further train an ALL DNN as an exemplary knowledge reserved network that is constrained to imitate the pretrained AML DNN. This knowledge reserved network may aim to predict MRD in the ALL database (or another target database associated with a different hematological malignancy) with parameters similar to those of the pretrained AML DNN.

In sum, the analysis platform may input AML representations and corresponding labels into a DNN (step 350). The analysis platform can then initialize the DNN and use AML data to predict class probabilities (step 351). Thereafter, the analysis platform can compute the derivative of loss function to update parameters of the DNN (step 352). Over time, these parameters may converge to stable values as discussed above. After observing loss convergence and then deriving the optimized parameters of the DNN (step 353), the analysis platform can obtain the pretrained AML DNN (step 354).

FIG. 4 includes a diagram illustrating how, with the parameters of the pretrained AML DNN 402 fixed at stable values, the representations and labels can be leveraged for the ALL database in training the knowledge reserved network 404. Much like FIG. 3, the process shown in FIG. 4 may be performed by the analysis platform as part of a knowledge distillation step (e.g., knowledge distillation step 104 of FIG. 1).

The knowledge reserved network 404 may be optimized with one or more loss functions. For example, the knowledge reserved network 404 may be optimized with two targeted losses, namely, ALL MRD classification loss and Kullback-Leibler divergence (KLD) loss. The KLD (also referred to as “relative entropy”) is a measure of how one probability distribution is different than another probability distribution (referred to as the “reference probability distribution”) that is known. Generally, MRD classification loss aims to minimize the error in the predictions produced by the pretrained AML DNN 402 given ground truth labels. Meanwhile, KLD loss can constrain learning of the parameters of the knowledge reserved network 404 to be similar to the pretrained AML DNN 402. These two losses can be summed up to jointly optimize the knowledge reserved network 404. In some embodiments, the knowledge reserved network 404 comprises a target MRD classification loss (e.g., an ALL MRD classification loss) and the KLD loss.

As shown in FIG. 4, this can be implemented by imposing the KLD loss between the parameters of the pretrained AML DNN 402 and the parameters of the knowledge reserved network 404. This approach to knowledge distillation may comprise distilling, filtering, or otherwise establishing knowledge in a network within the same classification task. In FIG. 4, for example, the capability to reserve compact knowledge is leveraged from AML data (i.e., source data) and then extended in a transfer learning scenario. In such a scenario, the knowledge reserved network 404 may keep the discriminative knowledge from the pretrained AML DNN 402, which relates to the MRD classification using the ALL data 406. This discriminative knowledge may be representative of the overlapping information between the hematological malignancy associated with the source domain and the hematological malignancy associated with the target domain. After the knowledge reserved network 404 has been optimized with the two losses, the analysis platform may extract one of the hidden layers in the knowledge reserved network 404 (e.g., the last layer) as a knowledge reserved embedding 408.

In sum, the analysis platform may input ALL representations and corresponding labels along with the parameters of the pretrained AML DNN into the knowledge reserved network 404 (step 450). The analysis platform can then initialize the knowledge reserved network 404 that has a comparable architecture as the pretrained AML DNN 402 (step 451). Generally, the knowledge reserved network 404 is representative of another DNN that is modeled after the pretrained AML DNN 402. Thus, the knowledge reserved network 404 may have the same architecture as the pretrained AML DNN 402. Thereafter, the analysis platform can compute the KLD loss between the parameters in the pretrained AML DNN 402 and the parameters in the knowledge reserved network 404 (step 452). The analysis platform can then optimize the knowledge reserved network 404 with the pair of losses, namely, the KLD loss and classification loss (step 453). After the knowledge reserved network 404 has been optimized, the analysis platform may extract the last hidden layer in the knowledge reserved network 404 as a knowledge reserved embedding 408 (step 454).

### C. Harmonized Learning

Although useful knowledge can be obtained from a pretrained source network (e.g., the pretrained AML DNN 402 of FIG. 4) in the knowledge distillation step, the knowledge reserved network (e.g., the knowledge reserved network 404 of FIG. 4) may completely miss the information that specifically exists in the target database (e.g., an ALL database, if knowledge is to be transferred from the AML domain to the ALL domain). While the pair of hematological malignancies associated with the source and target domains may have common immunophenotyping characteristics that are disclosed in the overlapping marker set, the natural difference in lineage suggests that deriving common information between the pair of hematological malignancies would leave out characteristics that are specific to the lineage of one hematological malignancy. As an example, if knowledge is distilled from AML data as discussed above for transfer to a model to be trained to classify MRD in ALL, then characteristics that are specific to ALL will simply be missed if the knowledge distillation relies entirely on characteristics shared between AML and ALL. To address this problem, the analysis platform may perform harmonized learning to elicit the ALL-specific knowledge that is omitted by the knowledge reserved network.

FIG. 5 includes a schematic flowchart of a process for performing harmonized learning. To accomplish this, the analysis platform may develop a harmonization network 502 that is designed to learn the residual of the knowledge reserved output 506. The harmonization network 502 may also be representative of a DNN. At a high level, the residual may be representative of the information missing from the knowledge reserved network. The harmonization network 502 may have two separate branches for inputs, a first branch for entry of ALL data 508 and a second branch for entry of a knowledge reserved embedding 510. The ALL data may be representative of target MRD input data, and the knowledge reserved embedding 510 may be derived by feeding the ALL data 508 to the knowledge reserved network and then extracting the last hidden layer as the embedding as discussed above with reference to FIG. 4. The harmonization network 502 may be able to predict ALL MRD ground truth labels and the residual, either sequentially or simultaneously. By computing the losses of these two predictions, the harmonization network 502 can be updated and then optimized as necessary.

To construct the harmonization network 502 with, for example, multiple layers between the input and output layers that are collectively representative of a DNN, the analysis platform can concatenate the latent layer with the knowledge reserved embedding 510 derived from the knowledge reserved network. The analysis platform can then train this harmonization network 502 to predict the residual between the ground truth and knowledge reserved output 506. The knowledge reserved output 506 may be representative of a prediction produced by the knowledge reserved network. As shown in FIG. 5, the predicted residual may be obtained by applying a loss to a target residual 504 that is representative of the difference between the ground truth and knowledge reserved output 506. As such, the harmonization network 502 can “fill the gap” between the ground truth and knowledge reserved output 506.

In sum, the analysis platform may input ALL representations and labels along with the knowledge reserved embedding 510 into the harmonization network 502 (step 550). The analysis platform can then initialize the harmonization network 502 as a complementary network with a pair of branches for inputs and a pair of branches for outputs (step 551). As shown in FIG. 5, the ALL representations and labels may be provided as a first input while the knowledge reserved embedding 510 may be provided as a second input. Meanwhile, the harmonization network 502 may produce a predicted class probability (e.g., corresponding to a proposed diagnosis) as a first output and a predicted residual as a second output. Then, the analysis platform can predict the ground truth and residual, either sequentially or simultaneously, and compute the corresponding loss to update the harmonization network 502 (step 552). Thereafter, the analysis platform may derive the converged complementary network that can be used to predict the residual (step 553).

### D. Classification Prediction

With the prediction of the knowledge reserved network and the predicted residual output by the harmonization network, the analysis platform can then make a final prediction. These values may be added or otherwise combined to obtain the final prediction. Accordingly, the final prediction may be representative of the combination of the knowledge reserved network guided by source domain knowledge (e.g., AML knowledge) and target domain-specific knowledge (e.g., ALL-specific knowledge) harmonizing to common information. Said another way, the final prediction may be indicative of the result of harmonizing source model knowledge and target-specific knowledge.

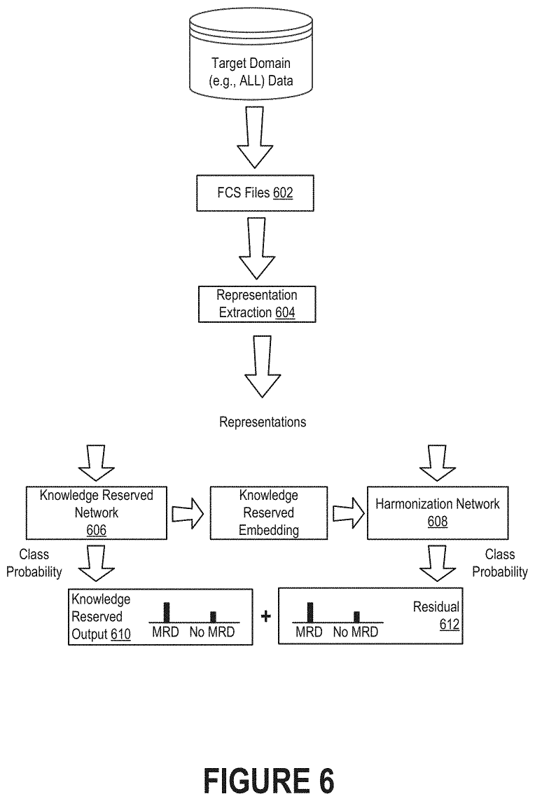

FIG. 6 includes a schematic diagram of the framework described above with reference to FIGS. 1-5 in its testing phase for the target domain. For the purpose of illustration, the framework is described in the context of FCS files 602 that contain information regarding ALL diagnoses, though the framework is similarly applicable to other hematological malignancies as mentioned above. In FIG. 6, FCS files 602 containing information regarding ALL diagnoses are initially encoded in the representation extraction stage 604. For example, an analysis platform may train a learning algorithm (e.g., GMM) as discussed above, and then each FCS file may be encoded as a vectorized representation using a vectorization algorithm (e.g., that implements Fisher scoring) that considers the parameters learned by the learning algorithm.

The analysis platform can then feed these vectorized representations into a knowledge reserved network 606 and a harmonization network 608 to obtain a knowledge reserved output 610 and a residual 612. At a high level, the knowledge reserved output 610 and residual 612 may be representative of class probability outputs. To derive the final classification output 614, the analysis platform may sum the knowledge reserved output 610 and residual 612.

### E. Use Case

The framework described herein was applied to a database managed by the National Taiwan University Hospital to illustrate its usefulness in transferring learning from one hematological malignancy to another. The database included FC data for patients who underwent bone marrow aspiration over the course of seven years. Each specimen was originally examined by one of two FC machines—FASCalibur and FASCanto from Becton Dickinson Bioscience. Two panels of fluorescent markers were used for AML (i.e., the source domain) and ALL (i.e., the target domain) clinical diagnoses to demonstrate how the approach may be used in the context of ALL, though the approach is similarly appliable to other hematological malignancies as mentioned above. The specimens were then judged by healthcare professionals as either normal (i.e., negative MRD) or abnormal (i.e., positive MRD). The training and testing phrases of this framework are shown in FIGS. 1 and 6, respectively.

The data for ALL corresponded to 493 patients, which resulted in a total of 2,356 unique specimens (279 positive MRD for ALL, 720 negative MRD for FASCalibur; 355 positive MRD for ALL, 1002 negative MRD for FASCanto). Meanwhile, the data for AML corresponded to 1,629 patients, which resulted in a total of 4,372 unique specimens (597 positive MRD for AML, 1,564 negative MRD for FASCalibur; 538 positive MRD for AML, 1,673 negative MRD for FASCanto).

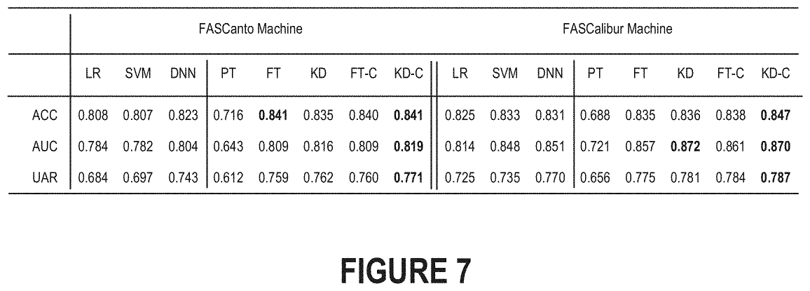

FIG. 7 includes the experimental results for three different classifiers, namely, Logistic Regression (LR), Support Vector Machine (SVM), and DNN. The term “PT” corresponds to the pretrained AML model that possesses discriminative capability, which means that some of all of the AML and ALL classification tasks may be transferable. A handful of transfer methods including fine tuning (FT), knowledge distillation (KD), and respective combinations with harmonized learning (FT-C and KD-C) were used. As can be seen in FIG. 7, knowledge distillation in combination with harmonized learning—indicated by KD-C—was able to achieve comprehensive improvements across the evaluated metrics. Note, however, that the approach to transfer learning appears to result in improved MRD classification in the target domain (e.g., ALL) regardless of the classifier and transfer method.

As noted above, although embodiments may be described in the context of a particular classifier (e.g., DNN) and transfer method (e.g., KD-C), those skilled in the art will recognize that the framework described herein may be similarly applicable regardless of the classifier and transfer method. As an example, the framework could rely on LR or SVM instead of DNN.

### Overview of Analysis Platform

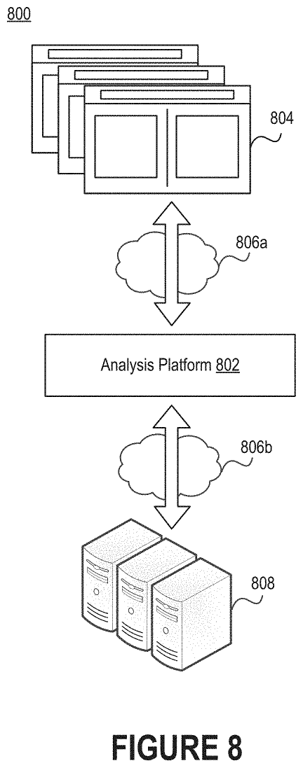

FIG. 8 illustrates a network environment 800 that includes an analysis platform 802. Individuals (also referred to as “users”) can interface with the analysis platform 802 via interfaces 804. For example, a user may be able to access an interface through which information regarding a patient, as well as a proposed diagnosis for the patient, can be viewed. These interfaces 804 may permit users to interact with the analysis platform 802 as it implements the framework described herein. The term “user,” as used herein, may refer to a person who is interested in examining a proposed diagnosis, such as a patient or healthcare professional, or a person who is interested in developing, training, or implementing models.

As shown in FIG. 8, the analysis platform 802 may reside in a network environment 800. Thus, the computing device on which the analysis platform 802 is implemented may be connected to one or more networks 806a-b. These networks 806a-b may be personal area networks (PANs), local area networks (LANs), wide area networks (WANs), metropolitan area networks (MANs), cellular networks, or the Internet. Additionally or alternatively, the analysis platform 802 can be communicatively coupled to one or more computing devices over a short-range wireless connectivity technology, such as Bluetooth®, Near Field Communication (NFC), Wi-Fi® Direct (also referred to as “Wi-Fi P2P”), and the like.

The interfaces 804 may be accessible via a web browser, desktop application, mobile application, or over-the-top (OTT) application. For example, a healthcare professional may be able to access an interface through which information regarding a patient can be input. Such information can include name, date of birth, symptoms, medications, and experiment results (e.g., in the form of an FCS file). With this information, the healthcare professional may be able to implement the framework to produce a classification that is representative of a proposed diagnosis. As another example, an individual may be access an interface through which she can identify the source domain (e.g., AML) and target domain (e.g., ALL) and monitor as the framework transfers learning from the source domain to the target domain. Accordingly, the interfaces 804 may be viewed on computing devices such as mobile workstations (also referred to as “medical carts”), personal computers, tablet computers, mobile phones, wearable electronic devices, and the like.

In some embodiments, at least some components of the analysis platform 802 are hosted locally. That is, part of the analysis platform 802 may reside on the computing device that is used to access the interfaces 804. For example, the analysis platform 802 may be embodied as a desktop application that is executable by a mobile workstation accessible to one or more healthcare professionals. Note, however, that the desktop application may be communicatively connected to a server system 808 on which other components of the analysis platform 802 are hosted.

In other embodiments, the analysis platform 802 is executed entirely by a cloud computing service operated by, for example, Amazon Web Services®, Google Cloud Platform™, or Microsoft Azure®. In such embodiments, the analysis platform 802 may reside on a server system 808 that is comprised of one or more computer servers. These computer servers can include models, algorithms (e.g., for processing data, generating reports, etc.), patient information (e.g., profiles, credentials, and health-related information such as age, date of birth, disease classification, healthcare provider, etc.), and other assets. Those skilled in the art will recognize that this information could also be distributed amongst the server system 808 and one or more computing devices. For example, some data that is generated by the computing device on which the analysis platform 802 resides may be stored on, and processed by, that computing device for security or privacy purposes.

### Methodologies for Transferring Learning Between Domains

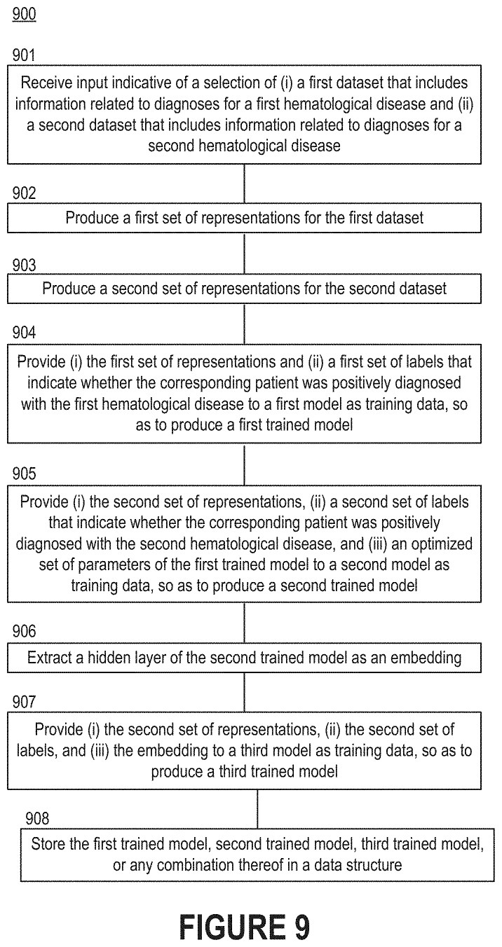

FIG. 9 includes a flow diagram of a process 900 for improving the classification of hematological malignancies (also referred to as “hematological diseases”) through transfer learning. At a high level, this process 900 attempts to address the gap between a first hematological disease (also referred to as a “source hematological disease”) and a second hematological disease (also referred to as a “target hematological disease”) by preserving knowledge of the source domain for better optimization of the target domain. As mentioned above, transfer learning may be appropriate if the first and second hematological diseases share at least one immunophenotyping characteristic in common. Harmonized learning, meanwhile, may also be performed to account for immunophenotyping characteristics that are unique to the second hematological disease (and thus cannot be learned from analysis of data associated with the first hematological disease).

Initially, an analysis platform may receive input indicative of a selection of (i) a first dataset that includes information related to diagnoses for a first hematological disease and (ii) a second dataset that information related to diagnoses for a second hematological disease (step 901). For example, the input may specify a first database in which the first dataset is stored in the form of FCS files and a second database in which the second dataset is stored in the form of FCS files. Alternatively, the input may specify a single database in which the first and second datasets are stored in the form of FCS files. Accordingly, the first and second datasets could be stored in separate databases (e.g., that are independently accessible by the analysis platform), or the first and second datasets could be stored in the same database.

The first dataset may include information regarding diagnoses for the first hematological disease for a first series of patients, while the second dataset may include information regarding diagnoses for the second hematological disease for a second series of patients. Each dataset could include information regarding positive and negative diagnoses. Accordingly, the diagnoses in the first dataset may include positive and negative diagnoses for the first hematological disease, and the diagnoses in the second dataset may include positive and negative diagnoses for the second hematological disease.

While each dataset will normally include information related to different series of patients, patients could be included in both sets. As an example, a given patient could be associated with a record of a negative diagnosis in the first dataset and a record of a positive diagnosis in the second dataset.

Then, the analysis platform can produce a first set of representations for the first dataset (step 902). To accomplish this, the analysis platform may extract a separate representation for each specimen in the first dataset. Assume, for example, that the first dataset includes FCS files that correspond to different FC experiments. In such a scenario, the analysis platform may apply a vectorization algorithm to a corresponding portion of the first dataset to produce a representation with fixed dimensions for each FCS file.

Similarly, the analysis platform can produce a second set of representations for the second dataset (step 903). To accomplish this, the analysis platform may extract a separate representation for each specimen in the second dataset. In the event that the second dataset includes FCS files that correspond to different FC experiments, the analysis platform may apply a vectorization algorithm to a corresponding portion of the second dataset to produce a representation with fixed dimensions for each FCS file. Generally, the same vectorization algorithm is applied in steps 902 and 903 to ensure that the representations have the same dimensions.

The analysis platform can then provide (i) the first set of representations and (ii) a first set of labels that indicate whether the corresponding patient was positively diagnosed with the first hematological disease to a first model as training data, so as to produce a first trained model (step 904). More specifically, the analysis platform can input (i) the first set of representations and (ii) the first set of labels into the first model for training purposes, and then the analysis platform can initialize the first model to predict class probabilities for the first set of representations. Said another way, the analysis platform can initialize the first model so that predictions representative of proposed diagnoses for the first hematological disease are produced based on the first set of representations. The analysis platform can then compute, based on the class probabilities, a loss function to update an initial set of parameters of the first model. These parameters may converge to stable values over time. These stable values may be representative of the optimized values for those parameters. Thus, the analysis platform can establish an optimized set of parameters by determining an optimized value for each parameter in the initial set of parameters. The analysis platform can then produce the first trained model by implementing the optimized set of parameters.

Thereafter, the analysis platform can provide (i) the second set of representations, (ii) a second set of labels that indicate whether the corresponding patient was positively diagnosed with the second hematological disease, and (iii) the optimized set of parameters of the first trained model to a second model as training data, so as to produce a second trained model (step 905). More specifically, the analysis platform can input (i) the second set of representations, (ii) the second set of labels, and (iii) the optimized set of parameters of the first trained model into the second model for training purposes, and then the analysis platform can initialize the second model to predict class probabilities for the second set of representations. Said another way, the analysis platform can initialize the second model so that predictions representative proposed diagnoses for the second hematological disease are produced based on the second set of representations. The analysis platform can then compute loss between the optimized set of parameters of the first trained model and an initial set of parameters of the second model. Based on this loss, the analysis platform can optimize the initial set of parameters to produce the second trained model.

The analysis platform can then extract a hidden layer of the second trained model as an embedding (step 906). For example, if the second trained model is representative of a DNN, the analysis platform may extract the last hidden layer as the embedding. The first trained model could also be a DNN with a comparable architecture.

Moreover, the analysis platform may provide (i) the second set of representations, (ii) the second set of labels, and (iii) the embedding to a third model as training data, so as to produce a third trained model (step 907). More specifically, the analysis platform can input (i) the second set of representations, (ii) the second set of labels, and (iii) the embedding into the third model for training purposes, and then the analysis platform can initialize the third model as a complementary model that is able to take (i) the second dataset and (ii) the embedding as input while producing (i) predicted classifications and (ii) predicted residuals as output. After initializing the third model, the analysis platform can compute loss between the predicted classifications and predicted residuals and then update, based on the loss, the third model to produce the third trained model.

The analysis platform can then store the first trained model, second trained model, third trained model, or any combination thereof in a data structure (step 908). As further discussed below, the analysis platform may subsequently use these models to produce classifications that are indicative of proposed diagnoses for the second hematological disease. As such, the analysis platform may programmatically associate these trained models with one another. For example, the analysis platform may store these trained models in the same data structure. As another example, the analysis platform may link these trained models together using, for example, an alphanumeric identifier.

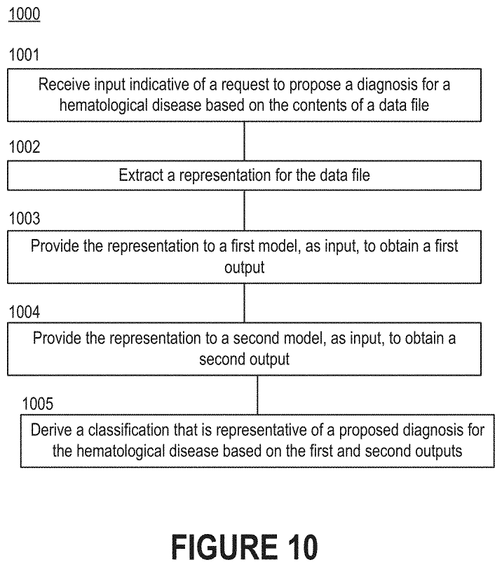

FIG. 10 includes a flow diagram of a process 1000 for effecting transfer learning by applying more than one trained model to data related to a specimen. Assume, for example, that an analysis platform receives input indicative of a request to propose a diagnosis for a hematological disease based on the contents of a data file (step 1001). This input may be representative of a selection of the file (or a corresponding patient) through an interface generated by the analysis platform, or this input may be representative of a receipt of the file (e.g., from an FC machine). The data file may be formatted in accordance with FCS, as an example.

In this situation, the analysis platform may extract a representation for the data file (step 1002). For example, the analysis platform may apply a vectorization algorithm to the data file to produce a representation with fixed dimensions. This vectorization algorithm may be the same vectorization algorithm discussed above with reference to steps 902-903 of FIG. 9.

The analysis platform can then provide the representation to a first model, as input, to obtain a first output (step 1003). This first model may be trained to produce outputs based on immunophenotyping characteristics that are shared between the hematological disease and another hematological disease. Thus, this first model may be the second model discussed above with reference to step 905 of FIG. 9. Moreover, the analysis platform can provide the representation to a second model, as input, to obtain a second output (step 1004). This second model may be trained to produce outputs based on immunophenotyping characteristics that are unique to the hematological disease. Thus, this second model may be the third model discussed above with reference to step 907 of FIG. 9.

The analysis platform can then derive a classification that is representative of a proposed diagnosis for the hematological disease based on the first and second outputs (step 1005). Generally, the first output is representative of a first class probability predicted by the first model, while the second output is representative of a second class probability predicted by the second model. As such, the analysis platform may derive the classification by summing, combining, or otherwise considering the first and second class probabilities. When applied to the representation, the first and second models may independently produce outputs that are indicative of class probabilities as mentioned above. While the output produced by the first model may be intended to account for immunophenotyping characteristics that are shared between the hematological disease and the other hematological disease, the output produced by the second model may be intended to account for immunophenotyping characteristics that are unique to the hematological disease.

Note that while the sequences of the steps in the processes described herein are exemplary, the steps can be performed in various sequences and combinations. For example, steps could be added to, or removed from, these processes. Similarly, steps could be replaced or reordered. Thus, the descriptions of these processes are intended to be open ended.

Additional steps may also be included in some embodiments. For example, the analysis platform may be able to derive a classification (e.g., a proposed diagnosis for a hematological disease) based on outputs produced by multiple models as discussed above. In such a scenario, the analysis platform may be able to cause display of the classification on an interface that is accessible to a patient associated with the underlying data. Similarly, the analysis platform may be able to cause display of the classification on an interface that is accessible to a healthcare professional. In some embodiments, the analysis platform is able to interface with the central computing system of a healthcare provider. For example, the analysis platform may be able to access the central computing system via a data interface (e.g., an application programming interface) to access FC data. In such a scenario, the analysis platform may be able to automatically populate the classification into the electronic health record (EHR) of the corresponding patient. For example, the analysis platform may transmit the classification to the central computing system with an instruction to populate the classification into the HER for recordation purposes.

### Processing System

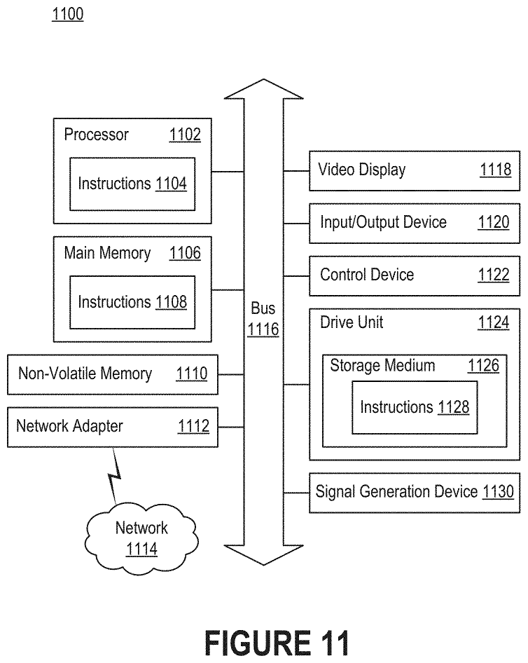

FIG. 11 is a block diagram illustrating an example of a processing system 1100 in which at least some operations described herein can be implemented. For example, components of the processing system 1100 may be hosted on a computing device that includes an analysis platform (e.g., analysis platform 802 of FIG. 8).

The processing system 1100 may include a processor 1102, main memory 1106, non-volatile memory 1110, network adapter 1112, video display 1118, input/output device 1120, control device 1122 (e.g., a keyboard, pointing device, or mechanical input such as a button), drive unit 1124 that includes a storage medium 1126, or signal generation device 1130 that are communicatively connected to a bus 1116. The bus 1116 is illustrated as an abstraction that represents one or more physical buses and/or point-to-point connections that are connected by appropriate bridges, adapters, or controllers. The bus 1116, therefore, can include a system bus, Peripheral Component Interconnect (PCI) bus, PCI-Express bus, HyperTransport bus, Industry Standard Architecture (ISA) bus, Small Computer System Interface (SCSI) bus, Universal Serial Bus (USB), Inter-Integrated Circuit (I²C) bus, or bus compliant with Institute of Electrical and Electronics Engineers (IEEE) Standard 1394.

The processing system 1100 may share a similar computer processor architecture as that of a computer server, router, desktop computer, tablet computer, mobile phone, video game console, wearable electronic device (e.g., a watch or fitness tracker), network-connected (“smart”) device (e.g., a television or home assistant device), augmented or virtual reality system (e.g., a head-mounted display), or another electronic device capable of executing a set of instructions (sequential or otherwise) that specify actions to be taken by the processing system 1100.

While the main memory 1106, non-volatile memory 1110, and storage medium 1124 are shown to be a single medium, the terms “storage medium” and “machine-readable medium” should be taken to include a single medium or multiple media that stores instructions. The terms “storage medium” and “machine-readable medium” should also be taken to include any medium that is capable of storing, encoding, or carrying instructions for execution by the processing system 1100.

In general, the routines executed to implement the embodiments of the present disclosure may be implemented as part of an operating system or a specific application, component, program, object, module, or sequence of instructions (collectively referred to as “computer programs”). Computer programs typically comprise instructions (e.g., instructions 1104, 1108, 1128) set at various times in various memories and storage devices in a computing device. When read and executed by the processor 1102, the instructions may cause the processing system 1100 to perform operations to execute various aspects of the present disclosure.

While embodiments have been described in the context of fully functioning computing devices, those skilled in the art will appreciate that the various embodiments are capable of being distributed as a program product in a variety of forms. The present disclosure applies regardless of the particular type of machine- or computer-readable medium used to actually cause the distribution. Further examples of machine- and computer-readable media include recordable-type media such as volatile and non-volatile memory devices 1110, removable disks, hard disk drives, optical disks (e.g., Compact Disk Read-Only Memory (CD-ROMS) and Digital Versatile Disks (DVDs)), cloud-based storage, and transmission-type media such as digital and analog communication links.

The network adapter 1112 enables the processing system 1100 to mediate data in a network 1114 with an entity that is external to the processing system 1100 through any communication protocol that is supported by the processing system 1100 and the external entity. The network adapter 1112 can include a network adaptor card, wireless network interface card, switch, protocol converter, gateway, bridge, hub, receiver, repeater, or transceiver that includes an integrated circuit (e.g., enabling communication over Bluetooth or Wi-Fi).

The techniques introduced here can be implemented using software, firmware, hardware, or a combination of such forms. For example, aspects of the present disclosure may be implemented using special-purpose hardwired (i.e., non-programmable) circuitry in the form of application-specific integrated circuits (ASICs), programmable logic devices (PLDs), field-programmable gate arrays (FPGAs), and the like.

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A non-transitory medium with instructions stored thereon that, when executed by a processor of a computing device, cause the computing device to perform operations comprising:
receiving input indicative of a selection of
(i) a first database in which first data related to acute myeloid leukemia (AML) diagnoses for a first series of patients is stored in the form of Flow Cytometry Standard (FCS) files, and
(ii) a second database in which second data related to acute lymphoblastic leukemia (ALL) diagnoses for a second series of patients is stored in the form of FCS files;
extracting
a separate representation for each FCS file in the first data to produce a first set of representations, and
a separate representation for each FCS file in the second data to produce a second set of representations;
providing (i) the first set of representations and (ii) a first of labels that indicate whether the corresponding patient was positively diagnosed with AML to a first model as training data, so as to produce a first trained model;
providing (i) the second set of representations, (ii) a second set of labels that indicate whether the corresponding patient was positively diagnosed with ALL, and (iii) an optimized set of parameters of the first trained model to a second model as training data, so as to produce a second trained model;
extracting a hidden layer of the second trained model as an embedding;
providing (i) the second set of representations, (ii) the second set of labels, and (iii) the embedding to a third model as training data, so as to produce a third trained model; and
storing the first, second, and third trained models in a data structure.

2. The non-transitory medium of claim 1, wherein said extracting comprises:
for each FCS file in the first data,
applying a vectorization algorithm to a corresponding portion of the first data to produce a representation with fixed dimensions, and
for each FCS file in the second data,
applying the vectorization algorithm to a corresponding portion of the second data to produce a representation with the same fixed dimensions.

3. The non-transitory medium of claim 1, wherein the first trained model is produced by:
inputting (i) the first set of representations and (ii) the first set of labels into the first model for training purposes,
initializing the first model to predict class probabilities for the first set of representations,
computing, based in the class probabilities, a loss function to update an initial set of parameters of the first model,
establishing the optimized set of parameters by determining an optimized value for each parameter in the initial set of parameters, and
producing the first trained model by implementing the optimized set of parameters.

4. The non-transitory medium of claim 1, wherein the second trained model is produced by:
inputting (i) the second set of representations, (ii) the second set of labels, and (iii) the optimized set of parameters of the first trained model for training purposes,
initializing the second model to predict class probabilities for the second set of representations,
computing loss between the optimized set of parameters of the first trained model and an initial set of parameters of the second model, and
optimizing, based on the loss, the initial set of parameters to produce the second trained model.

5. The non-transitory medium of claim 4, wherein the third trained model is produced by:
inputting (i) the second set of representations, (ii) the second set of labels, and (iii) the embedding for training purposes,
initializing the third model as a complementary model that is able to take (i) the second data and (ii) the embedding as input and produce (i) predicted classifications and (ii) predicted residuals as output,
computing loss between the predicted classifications and the predicted residuals, and
updating, based on the loss, the third model to produce the third trained model.

6. The non-transitory medium of claim 1, wherein the operations further comprise:
receiving input indicative of a request to propose an ALL diagnosis for a given FCS file;
extracting a representation for the given FCS file;
providing the representation to the second trained model, as input, to obtain a first output;
providing the representation to the third trained model, as input, to obtain a second output;
deriving a classification that is representative of a proposed diagnosis based on the first and second outputs.

7. The non-transitory medium of claim 6,
wherein the first output is representative of a first class probability predicted by the second trained model, and
wherein the second output is representative of a second class probability predicted by the third trained model.

8. The non-transitory medium of claim 7, wherein said deriving comprises:
summing the first and second class probabilities to obtain the classification.

9. The non-transitory medium of claim 6, wherein the operations further comprise:
causing display of the classification on an interface that is accessible to a patient associated with the given FCS file.

10. The non-transitory medium of claim 6, wherein the operations further comprise:
causing display of the classification on an interface that is accessible to a healthcare professional.

11. A method comprising:
producing a first set of representations by—
accessing a first dataset that includes information related to diagnoses for a first hematological disease, and
extracting a separate representation for each diagnosis;
producing a second set of representations by—
accessing a second dataset that includes information related to diagnoses for a second hematological disease, and
extracting a separate representation for each diagnosis;
providing the first set of representations to a first model as training data, so as to produce a first trained model;
providing (i) the second set of representations and (ii) parameters of the first trained model to a second model as training data, so as to produce a second trained model;
extracting a hidden layer of the second trained model as an embedding;
providing (i) the second set of representations and (ii) the embedding to a third model as training data, so as to produce a third trained model; and
storing the second and third trained models in a data structure.

12. The method of claim 11,
wherein the diagnoses in the first dataset include positive and negative diagnoses for the first hematological disease, and
wherein the diagnoses in the second dataset include positive and negative diagnoses for the second hematological disease.

13. The method of claim 11, wherein the first, second, and third models are representative of deep neural networks (DNNs).

14. The method of claim 11, wherein the first and second hematological diseases share at least one immunophenotyping characteristic in common.

15. The method of claim 11,
wherein when applied to a given representation, the second and third trained models produce outputs that are indicative of class probabilities,
wherein the output produced by the second trained model accounts for immunophenotyping characteristics that are shared by the first and second hematological diseases, and
wherein the output produced by the third trained model accounts for immunophenotyping characteristics that are unique to the second hematological disease.

16. The method of claim 11, wherein the first hematological disease is acute myeloid leukemia (AML), and wherein the second hematological disease is acute lymphoblastic leukemia (ALL).

17. The method of claim 11, wherein the first and second datasets are stored in separate databases.

18. The method of claim 11, further comprising:
receiving input indicative of a request to propose a diagnosis for the second hematological disease based on contents of a data file;
extracting a representation for the data file;
providing the representation to the second trained model, as input, to obtain a first output;
providing the representation to the third trained model, as input, to obtain a second output; and
deriving a classification that is representative of a proposed diagnosis based on the first and second outputs.

19. The method of claim 18, wherein the data file is formatted in accordance with the Flow Cytometry Standard (FCS).

20. A method comprising:
receiving input indicative of a request to propose a diagnosis for a hematological disease based on contents of a data file;
extracting a representation for the data file;
providing the representation to a first model, as input, to obtain a first output,
wherein the first model is trained to produce outputs based on immunophenotyping characteristics that are shared between the hematological disease and another hematological disease;
providing the representation to a second model, as input, to obtain a second output,
wherein the second model is trained to produce outputs based on immunophenotyping characteristics that are unique to the hematological disease; and
deriving, based on the first and second outputs, a classification that is representative of a proposed diagnosis for the hematological disease.
